(19) United States
(12) Patent Application Publication
(10) Pub. No.: US 2010/0143273 A1
(43) Pub. Date: Jun. 10, 2010

(54) COSMETIC COMPOSITION COMPRISING AT LEAST ONE APROTIC HYDROCARBON-BASED VOLATILE SOLVENT

(76) Inventors: Frédéric Auguste, Chevyilly-larue (FR); Jean-Yves Fouron, Bourg La Reine (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/516,015

(22) PCT Filed: Nov. 20, 2007
(86) PCT No.: PCT/EP2007/062583

§ 371 Date: Feb. 16, 2010

Related U.S. Application Data
(60) Provisional application No. 60/872,485, filed on Dec. 4, 2006.

(30) Foreign Application Priority Data
Nov. 23, 2006 (FR) 0655058

(51) Int. Cl. A61K 8/33; A61K 8/31; A61K 8/37; A61K 8/35; A61Q 19/10; A61Q 1/00; A61Q 5/00; A61Q 15/00; A61Q 5/02; A61Q 5/10; A61Q 17/04

(52) U.S. Cl. 424/59; 514/772; 514/785; 510/137; 510/159; 424/70.1; 424/65; 510/119; 8/405

(57) ABSTRACT

The present application relates to a cosmetic composition comprising, in a physiologically acceptable medium, at least one aprotic hydrocarbon-based volatile solvent comprising no more than one branch, the volatility of said solvent being such that the amount evaporated in 30 minutes is between 4.1 mg/cm² and 24 mg/cm². This composition can be used as a product for caring for and/or making up keratin materials, in particular the skin, the lips and/or the integuments.

The present invention relates to a cosmetic composition, in particular a cosmetic composition for making up or caring for the skin of the human face or body, the scalp, the lips or the integuments, such as the hair, the eyelashes, the eyebrows or the nails.

The composition of the invention can in particular constitute a care product, a hairstyling product or a make-up product for the lips, the body or the integuments that may also have care properties. The composition of the invention may in particular constitute a lipstick or a lip gloss, a face powder or an eye shadow, a tattoo product, a mascara, an eyeliner, an artificial skin-tanning product, a foundation or a care cream.

Cosmetic compositions must generally have certain properties such as staying power, migration resistance, transfer resistance, play-time, slip on application (or good spreading), comfort, sheen or coverage. The same composition does not necessarily have to have all these properties; however, in the majority of cases, it is desired for the composition to have at least some of them.

The staying power of the composition may in particular be the staying power with respect to water or to rubbing by the fingers, or alternatively with respect to tears, sweat or sebum.

The migration-resistance property corresponds, as far as the composition is concerned, to it not migrating into the folds of the skin, such as the wrinkles or fine lines located around the lips and the eyes (eyelids in particular).

The transfer-resistance characteristic of a composition corresponds to the fact that, once applied, it does not become notably deposited on the surfaces with which it comes into contact (glass, cup, cigarette, clothing, for example).

The play-time of a product corresponds to the time for which the consumer may work said product when applying it, and therefore reflects the ease with which the product is applied.

Cosmetic compositions commonly comprise a fatty phase containing a volatile solvent. In fact, said solvent makes it possible to bring about a change in the properties of the product during and after deposition, thereby resulting, depending on the cosmetic product envisaged, in properties of staying power of the deposited product, or of comfort or texture during application of the product, and also in specific mechanical or optical properties of the deposits.

These volatile solvents are conventionally used in the care, hygiene, hair product, fragrance and make-up fields, in very varied galenical forms: direct or inverse emulsions, anhydrous pastes, anhydrous sticks, solid emulsions, etc.

In the context of the formulation of cosmetic compositions, it would be advantageous to have novel volatile solvents which make it possible to obtain compositions having at least some of the properties stated above.

The Applicant has discovered, unexpectedly, that a specific category of compounds meets the criteria stated above and thus confers on the compositions excellent cosmetic properties such as good spreading and/or a non-greasy feel and/or comfort and/or transfer resistance and/or migration resistance, for example.

This list is not exhaustive and, more generally, these compounds exhibit good compatibility with other constituents normally present in cosmetic compositions and confer most of the conventionally desired properties on the composition.

More specifically, a subject of the invention is a cosmetic composition comprising, in a physiologically acceptable medium, at least one aprotic hydrocarbon-based volatile solvent comprising no more than one branch, the volatility of said solvent being such that the amount evaporated in 30 minutes is between 4.1 mg/cm² and 24 mg/cm² (when it is measured on the compound alone, under the conditions defined hereinafter).

The measurement of the volatility of a solvent is described in Patent Application WO 06/013413, as a function of the amount evaporated in 30 minutes, according to the protocol defined hereinafter:

### Measurement of the Rate of Evaporation of a Solvent (Protocol)

15 g of oil or of the mixture of oils to be tested are placed in a crystallizing dish (diameter: 7 cm) placed on a balance that is inside a chamber of about 0.3 m³ with a regulated temperature (25° C.) and a regulated hygrometry (relative humidity 50%). The liquid is allowed to evaporate freely, without stirring it, while providing ventilation with a fan (Papst-Motoren, reference 8550 N, rotating at 2700 rpm) positioned vertically above the crystallizing dish containing the solvent, the vanes facing the crystallizing dish and being 20 cm from the bottom of the crystallizing dish. The mass of oil remaining in the crystallizing dish is measured at regular intervals. The evaporation rates are expressed in mg of oil evaporated per unit of surface area (cm²) and per unit of time (minutes).

By way of indication, the amount by mass of volatile oil evaporated after 30 minutes for certain volatile oils (not corresponding to the definition of hydrocarbon-based volatile solvent comprising no more than one branch) according to this protocol is given hereinafter:
isododecane: 24 mg/cm²,
octamethylcyclotetrasiloxane (D4): 18.7 mg/cm²,
decamethylcyclohexasiloxane (D5): 4.1 mg/cm².

Preferably, said hydrocarbon-based volatile solvent is aprotic.

The term “aprotic solvent” is intended to mean a compound comprising few or no hydrogen atoms linked to a heteroatom, or more generally to a highly electro-negative atom such as O, N or Si. In particular, the term “aprotic oil” is intended to mean oils which can comprise, depending on the yield from their synthesis, residual groups carrying a labile hydrogen atom (for example, residual OH, NH and/or COOH groups) in an amount of less than or equal to 5% by number.

The expression “a hydrocarbon-based solvent” covers the case where one or more individual compounds is (are) involved, and also the mixture thereof. Thus, this expression covers “at least one hydrocarbon-based solvent”.

The term “hydrocarbon-based” is intended to mean a radical or a compound formed essentially of or even consisting of carbon and hydrogen atoms, and optionally of oxygen, nitrogen, sulphur or phosphorus atoms, and containing no silicon or fluorine atoms. It may contain alcohol, ether, carboxylic acid, amine and/or amide groups. Preferably, the adjective “hydrocarbon-based” denotes a radical or a compound consisting only of carbon and hydrogen atoms, such as alkyl, alkenyl or alkynyl radicals, for example.

The number of branches of a hydrocarbon-based radical corresponds to the number of —CH₃ groups on the whole molecule, minus one (which corresponds to the terminal CH₃ of the main chain). More generally, the number of branches of a molecule corresponds to the number of side groups containing at least one carbon atom and branched on the main chain of the molecule, the main chain corresponding to the longest carbon chain of the molecule (see Organic Chemistry, S. H. Pine, 5th edition; McGraw-Hill, chapter 3).

In the context of the present invention, only compounds in which the whole molecule exhibits no more than one branch are taken into account.

Preferably, the composition according to the invention is a cosmetic composition for making up or caring for keratin materials.

In the context of the present invention, the term “keratin materials” comprises the skin, the lips, the nails, the hair, the eyelashes and the eyebrows, and the term “keratin fibres” comprises the hair, the eyelashes and the eyebrows.

Advantageously, said hydrocarbon-based volatile solvents comprise at least one oxygen atom. The volatile solvents may thus be, for example, esters, ethers, carbonic acid esters, ketones or aldehydes.

Preferably, said hydrocarbon-based volatile solvents contain a minimum of 7 carbon atoms.

Advantageously, said hydrocarbon-based volatile solvents contain a maximum of 13 carbon atoms.

Preferably, said hydrocarbon-based volatile solvents are linear; i.e. the hydrocarbon-based radical(s) of the compound is (are) linear. When said hydrocarbon-based volatile solvents are linear, they advantageously contain between 7 and 11 carbon atoms.

Preferably, said hydrocarbon-based volatile solvents are saturated.

Advantageously, the hydrocarbon-based radical(s) of said volatile solvents is (are) aliphatic.

Advantageously, the hydrocarbon-based radical(s) of said volatile solvents is (are) alkyl(s).

Preferably, said volatile solvents are lipophilic.

The term “lipophilic” is intended to mean water-immiscible solvents. The lipophilic solvents are defined on the basis of their solubility parameter δa which is given by the following equation:

δₐ=\sqrt{δₚ²+δₕ²}

where δp and δh are the Hansen solubility parameters calculated using group contributions, according to the reference “Van Krevelen, D. W., Properties of Polymer: Their Correlation with Chemical Structure; Their Estimation and Prediction from Additive Group Contribution. 3rd ed. Elsevier (1990)”. The calculations are given in chapter 7 of said work. The equations giving the solubility parameters δ_{d}, δₚ and δₕ are given on page 212 (Hoftyzer & Van Krevelen method). They are calculated from the molar volume of the desired constituent, which is given by Table 7.9, page 215 (V=ΣNᵢVᵢ) and from the values of Vᵢ appearing in Table 7.3, pages 196-197. The number Nᵢ represents the number of groups i per molecule. The equations also involve parameters F_{di}, Fₚᵢ and Eₕᵢ, which are given by Table 7.8, page 213.

The lipophilic solvents according to the present invention are considered to have values of δa<15 J^{1/2}·cm^{-3/2}, better still δa<10 J^{1/2}·cm^{-3/2}.

Preferably, said volatile solvents have a flash point of between 43 and 100° C., and more particularly between 45 and 80° C. By way of indication, the flash points of isododecane and of the cyclomethicone D5 are 45° C. and 77° C., respectively.

Preferably, said volatile solvents have a surface tension of less than 30 mN/m.

Advantageously, said volatile solvents have a viscosity of less than 10 mPa·s.

Preferably, said volatile solvents are miscible in any proportions with parleam and isononyl isononanoate and have a water-miscibility of less than 5%.

Advantageously, said volatile solvents are in liquid form between 4° C. and 100° C.

Preferably, said volatile solvents are miscible in all proportions with hydrogenated polyisobutene and isononyl isononanoate.

Advantageously, the volatile solvents according to the invention have a water-miscibility of less than 5%.

Preferably, said volatile solvents are in liquid form between 4° C. and 100° C.

The hydrocarbon-based volatile solvents (comprising no more than one branch) of the compositions according to the invention are, for example:

1) Esters of formula R1COOR2, in which R1 represents a hydrogen atom H or a linear or branched hydrocarbon-based radical, and R2 represents a linear or branched hydrocarbon-based radical, with the proviso that:
when R2 is a linear hydrocarbon-based radical and R1 is H or a linear hydrocarbon-based radical, then 7≦R1+R2≦8; and
when at least one of R1 and R2 is a branched hydrocarbon-based radical, then 8≦R1+R2≦10.
   2) ketones of formula R1-CO—R2 in which R1 and R2 are identical or different and represent a linear or branched hydrocarbon-based radical, with the proviso that:
when R1 and R2 are linear, they each contain from 1 to 8 carbon atoms, with 8≦R1+R2≦9; and
when R1 and/or R2 are branched, they each contain from 1 to 10 carbon atoms, with 9≦R1+R2≦11.
   3) ethers of formula R1-O—R2, in which R1 and R2 are identical or different and represent a linear or branched hydrocarbon-based radical, with the proviso that:
when R1 and R2 are linear, they each contain from 1 to 10 carbon atoms, with 10≦R1+R2≦11; and
when R1 and/or R2 are branched, they each contain from 1 to 12 carbon atoms, with 10≦R1+R2≦13, and preferably with 12≦R1+R2≦13.

According to a first embodiment, the ethers of formula R1-O—R2 are those wherein R1+R2=10

According to a second embodiment, the ethers of formula R1-O—R2 are those wherein R1+R2=11

According to a third embodiment, the ethers of formula R1-O—R2 are those wherein R1+R2=12

According to a fourth embodiment, the ethers of formula R1-O—R2 are those wherein R1+R2=13

4) carbonic acid esters of formula R1-O—CO—O—R2, in which R1 and R2 are identical or different and represent a linear or branched hydrocarbon-based radical, with the proviso that:
when R1 and R2 are linear, they each contain from 1 to 6 carbon atoms, with 6≦R1+R2≦7; and
when R1 and/or R2 are branched, they each contain from 1 to 8 carbon atoms, with 6≦R1+R2≦9.
   5) aldehydes of formula RICOH, in which R1 represents a linear or branched hydrocarbon-based radical, with the proviso that:
when R1 is linear, R1 contains 7 or 8 carbon atoms; and
when R1 is branched, R1 contains 8 to 10 carbon atoms.

In the various formulae targeted in points 1) to 4), R1 and R2 (when it exists) are chosen independently of one another and are two distinct radicals, i.e. they are not linked to one another by a covalent bond.

As indicated above, preferably, R1 and/or R2 (when the latter exists) are preferably aliphatic hydrocarbon-based radicals, and in particular alkyls.

According to a first embodiment, R1 and/or R2 (when the latter exists) are linear radicals.

According to an other embodiment, one of R1 and R2 (when the latter exists) is a branched radical.

According to an advantageous embodiment, the hydrocarbon-based volatile solvents does not comprise a quaternary carbon.

By way of example of esters according to the invention, mention may be made of:
pentyl propanoate,
ethyl hexanoate
heptyl formate,
butyl butanoate,
methyl heptanoate,
hexyl acetate,
propyl pentanoate,
ethyl heptanoate,
methyl octanoate,
heptyl acetate,
octyl formate,
hexyl propanoate,
pentyl butanoate,
butyl pentanoate,
propyl hexanoate,
1-methylethyl hexanoate,
1-methylethyl heptanoate,
1-methylethyl octanoate,
2-pentyl butyrate,
isoamyl butyrate,
hexyl isobutyrate.

By way of example of ketones according to the invention, mention may be made of:
4-nonanone,
5-nonanone,
2-nonanone,
3-nonanone,
4-decanone,
5-decanone,
2-decanone,
3-decanone,
3-octanone, 7-methyl-,
3-nonanone, 7-methyl-,
2-decanone, 8-methyl-,
isododecanone.

By way of example of ethers according to the invention, mention may be made of:
nonane, methoxy-;
pentane, 1,1′-oxybis-;
heptane, 1-propoxy-;
nonane, 1-methoxy-;
octane, 1-ethoxy-;
nonane, 1-ethoxy-;
octane, 1-propoxy-;
hexane, 1-(pentyloxy);
heptane, 1-butoxy-;
decane, 1-methoxy-;
octane, 2-ethoxy-;
nonane, 4-ethoxy-;
decane, 2-ethoxy;
ether, isopentyl octyl.

By way of example of carbonic acid esters according to the invention, mention may be made of:
methyl n-pentyl carbonate;
n-butyl ethyl carbonate;
dipropyl carbonate;
ethyl n-pentyl carbonate;
n-butyl n-propyl carbonate;
n-hexyl methyl carbonate;
1-methylethyl n-propyl carbonate;
ethyl 1-ethylpropyl carbonate;
ethyl 1-methylpentyl carbonate;
isooctyl methyl carbonate;
1,1-diethylpropyl methyl carbonate.

By way of example of aldehydes according to the invention, mention may be made of:
octanal,
nonanal,
7-methyloctanal,
2-methyloctanal,
5-ethylheptanal,
2-ethylheptanal,
8-methylnonanal,
2-methylnonanal,
6-ethyloctanal,
2-ethyloctanal,
2,4-diethylhexanal,
9-methyldecanal,
2-methyldecanal,
5-ethylnonanal,
2-ethylnonanal,
8,8-dimethylnonanal,
3,5-dimethylheptanal.

A second subject of the invention is a cosmetic composition comprising, in a physiologically acceptable medium, at least one aprotic hydrocarbon-based volatile solvent, the volatility of said solvent being such that the amount evaporated in 30 minutes is between 4.1 mg/cm² and 24 mg/cm² (when it is measured on the compound alone, under the conditions defined hereinbefore, wherein said hydrocarbon-based volatile solvent is an ether of formula (I)

R1-O—R2 (I)

in which R1 and R2 are identical or different and represent a linear or branched hydrocarbon-based radical, with the proviso that:
when R1 and R2 are linear, they each contain from 1 to 10 carbon atoms, with 10≦R1+R2≦11; and
when R1 and/or R2 are branched, they each contain from 1 to 12 carbon atoms, with 10≦R1+R2≦13, and preferably with 12≦R1+R2≦13.

According to this second aspect of the invention, the following embodiments can be used:

According to a first embodiment, the ethers of formula R1-O—R2 are those wherein R1+R2=10

According to a second embodiment, the ethers of formula R1-O—R2 are those wherein R1+R2=11

According to a third embodiment, the ethers of formula R1-O—R2 are those wherein R1+R2=12

According to a fourth embodiment, the ethers of formula R1-O—R2 are those wherein R1+R2=13

Preferably, R1 and/or R2, identical or different, are preferably aliphatic hydrocarbon-based radicals.

Preferably, R1 and/or R2, identical or different, are alkyls.

According to a first embodiment, R1 and/or R2 are linear radicals.

According to a second embodiment, R1 and/or R2 are branched radicals.

According to an advantageous embodiment, R1 and/or R2 does not comprise a quaternary carbon.

Preferably, the ether of formula I does not comprise no more than four branches, or better, no more than 2 or 3 branches.

Preferably, the ether of formula I does not comprise no more than one branch.

By way of example of ethers according to the second object of the invention and to the first object of the invention (ether comprising no more than one branch, mention may be made of:

Linear ether comprising 10 carbon atoms:
Nonane, methoxy-;
Pentane, 1,1′-oxybis;
Heptane, 1-propoxy-;
Nonane, 1-methoxy-;
Octane, 1-ethoxy-.

Linear ether comprising 11 carbon atoms:
Nonane, 1-ethoxy-;
Octane, 1-propoxy-;
Hexane, 1-(pentyloxy);
Heptane, 1-butoxy-;
Decane, 1-methoxy-.

Among the branched esters containing 10 carbon atoms, mention may be made of:
Hexane, 2-butoxy-;
Butane, 2,2′-oxybis[3-methyl-;
Pentane, 1-(1,1-dimethylpropoxy)-;
Pentane, 1-[1-methylbutoxy]-;
Pentane, 1-[1-methylbutoxy]-;
Ether, 1-ethyl-1,2-dimethylpentyl methyl;
Pentane, 2-(1-ethylpropoxy)-;
Pentane, 1-(2,2-dimethylpropoxy)-;
Pentane, 2-(2,2-dimethylpropoxy)-;
Pentane, 2-ethoxy-2,4,4-trimethyl-;
Nonane, 4-methoxy-;
Hexane, 3-ethoxy-2,5-dimethyl-;
Hexane, 2-(1,1-dimethylethoxy)-;
Butane, 3-(1,1-dimethylethoxy)-2,2-dimethyl-;
Propane, 1,1′-oxybis[2,2-dimethyl-;
Ether, bis(2-methylbutyl);
Ether, 2-methylbutyl pentyl;
Ether, 2-methylbutyl pentyl;
Heptane, 2-propoxy-;
Hexane, 2-methyl-2-propoxy-;
Octane, 2-ethoxy-;
Pentane, 2-(1,1-dimethylethoxy)-2-methyl-;
Butane, 1-(1,1-dimethylethoxy)-3,3-dimethyl-;
Nonane, 5-methoxy-;
Butane, 1,1′-oxybis[3-methyl-;
Nonane, 2-methoxy-;
Heptane, 3-(ethoxymethyl)-;
Pentane, 3,3′-oxybis-;
Pentane, 2,2′-oxybis-;
Heptane, 1-methoxy-6,6-dimethyl-;
Octane, 3-ethoxy-;
Octane, 2-methoxy-2-methyl-;
Heptane, 4-ethyl-4-methoxy-;
Hexane, 3-ethoxy-3-ethyl-;
Octane, 2-ethoxy-;
Heptane, 2-methoxy-2,6-dimethyl-;
Hexane, 3-ethyl-2-methoxy-2-methyl-;
Hexane, 1-(1-methylpropoxy)-;
Butane, 2,2′-oxybis[2-methyl-;
Butane, 2-methyl-2-(3-methylbutoxy)-;
Hexane, 1-(1,1-dimethylethoxy)-;
Butane, 2-methyl-1-(3-methylbutoxy)-;
Heptane, 3-methoxy-2,3-dimethyl-;
Heptane, 2-methoxy-2,3-dimethyl-;
Octane, 4-ethoxy-;
Heptane, 4-methoxy-3,5-dimethyl-;
Ether, 1,2-dimethylhexyl ethyl;
Pentane, 1-(1-methylbutoxy)-;
Pentane, 1-(2-methylbutoxy)-;
Pentane, 2-(2-methylbutoxy)-;
Pentane, 1-ethoxy-2,4,4-trimethyl-;
Ether, ethyl 2-propylamyl;
Ether, methyl β-methyloctyl;
Ether, 1-ethylheptyl methyl;
Ether, ethyl 3-methylheptyl;
Heptane, 1-(1-methylethoxy)-;
Heptane, 3-ethyl-3-methoxy-;
Pentane, 3-butoxy-2-methyl-;
Pentane, 2-butoxy-4-methyl-;
Ether, 1-tert-butylpentyl methyl;
Ether, 1-isobutyl-3-methylbutyl methyl;
Ether, propyl 1-propylbutyl;
Hexane, 1-(2-methylpropoxy)-;
Pentane, 1-(3-methylbutoxy)-;
Ether, methyl 2-propylhexyl.

Among the branched esters containing 11 carbon atoms, mention may be made of:
Ether, methyl 1,1,5-trimethylheptyl;
Ether, butyl 1,4-dimethylpentyl;
Pentane, 2-(1,2,2-trimethylpropoxy)-;
Pentane, 2-(1,2,2-trimethylpropoxy)-;
Heptane, 2-ethoxy-2,6-dimethyl-;
Hexane, 1-(1,1-dimethylpropoxy)-;
Hexane, 2-(pentyloxy)-;
Hexane, 3-(pentyloxy)-;
Hexane, 2-(pentyloxy)-;
Hexane, 2-(pentyloxy)-;
Hexane, 3-(pentyloxy)-;
Hexane, 3-(pentyloxy)-;
Hexane, 1-(1,1-dimethylethoxy)-4-methyl-;
Pentane, 1-(1,1-dimethylethoxy)-4,4-dimethyl-;
Heptane, 3-methoxy-2,4,6-trimethyl-;
Heptane, 3-methoxy-2,4,6-trimethyl-;
Heptane, 4-ethoxy-2,6-dimethyl-;
Nonane, 4-ethoxy-;
Octane, 6-methoxy-2,6-dimethyl-;
Octane, 6-methoxy-2,6-dimethyl-;
Hexane, 1-(2-methylbutoxy)-;
Ether, hexyl 2-methylbutyl;
Heptane, 3-methoxy-2,2,3-trimethyl-;
Pentane, 4-methyl-1-(3-methylbutoxy)-;
Decane, 2-methoxy-;
Hexane, 4-ethyl-4-methoxy-2,2-dimethyl-;
Pentane, 2,2,4-trimethyl-4-propoxy-;
Pentane, 2,2,4-trimethyl-4-(1-methylethoxy)-;
Heptane, 3-(methoxymethyl)-2,6-dimethyl-;
Ether, 2,2-dimethylbutyl tert-pentyl;
Hexane, 1-ethoxy-3-ethyl-5-methyl-;
Hexane, 2-ethoxy-2,4,4-trimethyl-;
Decane, 3-methoxy-;
Hexane, 4-butoxy-2-methyl-;
Nonane, 3-methoxy-3-methyl-;
Octane, 3-ethyl-3-methoxy-;
Octane, 4-ethyl-4-methoxy-;
Nonane, 5-methoxy-5-methyl-;
Octane, 1-(1-methylethoxy)-;
Heptane, 3-(1,1-dimethylethoxy)-;
Nonane, 2-methoxy-2-methyl-;
Heptane, 1-(1,1-dimethylethoxy)-;
Hexane, 3-methoxy-2,2,5,5-tetramethyl-;
Hexane, 2-methoxy-2,3,5,5-tetramethyl-;
Hexane, 2-methyl-2-(2-methylpropoxy)-;
Ether, heptyl isobutyl;
Ether, ethyl 1-ethylheptyl;
Ether, methyl 5-methyl-3-propylhexyl;
Ether, (-methylheptyl propyl;
Octane, 1-methoxy-2,6-dimethyl-;
Octane, 1-methoxy-3,6-dimethyl-;
Octane, 1-methoxy-2,7-dimethyl-;
Ether, 1-butylpentyl ethyl;
Octane, 6-methoxy-2,6-dimethyl-;
Nonane, 2-ethoxy-;
Octane, 1-methoxy-3,7-dimethyl-;
Heptane, 3-[(1-methylethoxy)methyl]-;
Pentane, 3-(methoxymethyl)-2,2,4,4-tetramethyl-;

Among the branched esters containing 12 carbon atoms, mention may be made of:
Ether, 1,3-diethylheptyl methyl;
Butane, 2,2′-oxybis[3,3-dimethyl-;
Octane, 2-butoxy-;
Octane, 1-ethoxy-2,6-dimethyl-;
Undecane, 6-methoxy-;
Octane, 6-ethoxy-2,6-dimethyl-;
Octane, 2-ethoxy-2,6-dimethyl-;
Nonane, 1-(1-methylethoxy)-;
Nonane, 2-(1-methylethoxy)-;
Pentane, 3,3′-[oxybis(methylene)]bis-;
Pentane, 2-butoxy-2,4,4-trimethyl-;
Heptane, 1-(1,1-dimethylpropoxy)-;
Octane, 2-(1-methylpropoxy)-;
Pentane, 1,1′-oxybis[2-methyl-;
Pentane, 2,2′-oxybis[4-methyl-;
Octane, 1-ethoxy-3,7-dimethyl-;
Octane, 6-ethoxy-2,6-dimethyl-;
Octane, 6-ethoxy-2,6-dimethyl-;
Heptane, 1-(2-methylbutoxy)-;
Ether, heptyl 2-methylbutyl;
Undecane, 3-methoxy-;
Pentane, 2,2,4-trimethyl-4-(2-methylpropoxy)-;
Pentane, 2,2,4-trimethyl-4-(1-methylpropoxy)-;
Isohexane, oxybis-;
Heptane, 1-(1,1-dimethylethoxy)-3-methyl-;
Octane, 1-(1-methylpropoxy)-;
Octane, 1-(1-methylpropoxy)-;
Octane, 1-(1,1-dimethylethoxy)-;
Decane, 2-ethoxy-;
Hexane, 2,2′-oxybis-;
Hexane, 1-butoxy-2,2-dimethyl-;
Heptane, 3-(butoxymethyl)-;
Nonane, 3-ethyl-3-methoxy-;
Nonane, 4-ethyl-4-methoxy-;
Decane, 4-methoxy-4-methyl-;
Octane, 4-methoxy-4-propyl-;
Pentane, 1,1′-oxybis[4-methyl-;
Pentane, 2,2′-oxybis[2-methyl-;
Pentane, 3,3′-oxybis[3-methyl-;
Butane, 1,1′-oxybis[2,2-dimethyl-;
Hexane, 2,2′-oxybis-;
Hexane, 2,2′-oxybis-;
Octane, 1-ethoxy-3,7-dimethyl-;
Octane, 1-ethoxy-3,7-dimethyl-;
Nonane, 5-(ethoxymethyl)-;
Hexane, 1-[(1-methylpentyl)oxy]-;
Hexane, 1-(1-ethylbutoxy)-;
Pentane, 1,1′-oxybis[3-methyl-;
Heptane, 3-[(1,1-dimethylethoxy)methyl]-;
Octane, 2-(1,1-dimethylethoxy)-;
Heptane, 2-(pentyloxy)-;
Heptane, 2-(pentyloxy)-;
Heptane, 2-(pentyloxy)-;
Ether, heptyl isoamyl;
Ether, 4,8-dimethylnonyl methyl;
Ether, 3-isobutylheptyl methyl;
Ether, (-ethylheptyl propyl;
Ether, butyl 3-methylheptyl;
Octane, 1-(2-methylpropoxy)-;
Ether, 1-butylhexyl ethyl;
Ether, ethyl 1-ethyloctyl;
Ether, ethyl 1-propylheptyl;
Undecane, 2-methoxy-;
Butane, 1,1′-oxybis[3,3-dimethyl-.

Among the branched esters containing 13 carbon atoms, mention may be made of:
Hexane, 5-methyl-2-(1,2,2-trimethylpropoxy)-;
Hexane, 5-methyl-2-(1,2,2-trimethylpropoxy)-;
Heptane, 3-[(pentyloxy)methyl]-;
Heptane, 4-butoxy-2,2-dimethyl-;
Nonane, 2-(1,1-dimethylethoxy)-;
Nonane, 2-methyl-2-(1-methylethoxy)-;
Octane, 1-(2-methylbutoxy)-;
Octane, 1-(1,1-dimethylpropoxy)-;
Heptane, 1-(1,1-dimethylethoxy)-2,2-dimethyl-;
Octane, 1-(2,2-dimethylpropoxy)-;
Hexane, 2,5-dimethyl-3-(3-methylbutoxy)-;
Ether, 2-methylbutyl octyl, (−)-;
Heptane, 4-methoxy-2,2,4,6,6-pentamethyl-;
Octane, 2-methoxy-2,3,5,7-tetramethyl-;
Pentane, 2,2,4-trimethyl-4-(pentyloxy)-;
Nonane, 1-(1-methylpropoxy)-;
Octane, 1-(1-methylbutoxy)-;
Octane, 2-(pentyloxy)-;
Nonane, 1-(1-methylpropoxy)-;
Octane, 1-(1-methylbutoxy)-;
Octane, 2-(pentyloxy)-;
Heptane, 1-(1,1-dimethylethoxy)-2,6-dimethyl-;
Octane, 1-(1-ethylpropoxy)-;
Undecane, 5-methoxy-5-methyl-;
Nonane, 3-ethoxy-3-ethyl-;
Decane, 4-ethyl-4-methoxy-;
Nonane, 5-methoxy-5-propyl-;
Nonane, 1-(1,1-dimethylethoxy)-;
Heptane, 2-methoxy-2,4,4,6,6-pentamethyl-;
Ether, 2,2-dimethyldecyl methyl;
Hexane, 1-(hexyloxy)-2-methyl-;
Hexane, 1-(hexyloxy)-3-methyl-;
Hexane, 1-(hexyloxy)-5-methyl-;
Hexane, 1-(hexyloxy)-4-methyl-;
Heptane, 2-(hexyloxy)-;
Heptane, 2-[(1-methylpentyl)oxy]-;
Heptane, 1-(1,1-dimethylethoxy)-2,6-dimethyl-;
Octane, 2-(pentyloxy)-;
Ether, isopentyl octyl;
Ether, butyl α-ethylheptyl;
Decane, 2-(1-methylethoxy)-;
Dodecane, 5-methoxy-;
Octane, 2,6-dimethyl-1-propoxy-;
Octane, 3,6-dimethyl-1-propoxy-;
Octane, 2,7-dimethyl-1-propoxy-;
Undecane, 2-methoxy-2-methyl-;
Ether, 2-methylbutyl octyl;
Dodecane, 2-methoxy-.

The volatile solvent(s) according to the invention can be used as sole volatile lipophilic solvent or as a mixture with other additional lipophilic volatile solvents (also known as “oils”) which do not correspond to the definition of the lipophilic solvents according to the invention.

In the case of a mixture of volatile solvents, the measuring protocol is the same as that described above.

### Equations Used:

Taking 100 mg of a composition comprising i solvents each having an evaporation rate vᵢ (measured according to the protocol described above), expressed as mg of oil evaporated per unit of surface area (cm²) and per unit of time (minute).

The solvents are introduced into the composition in an initial amount per unit of surface area equal to mᵢ(0) (expressed in mg per cm²).

For each solvent, the remaining mass per unit of surface area at a time t [mᵢ(t)] can be given by the following equations:

$${m}_{i} ( t ) = {m}_{i} ( 0 ) - {v}_{i} \cdot t if t < \frac{{m}_{i} ( 0 )}{{v}_{i}}$$

$${m}_{i} ( t ) = 0 if t \geq \frac{{m}_{i} ( 0 )}{{v}_{i}}$$

The total mass of liquid fatty phase can then be given by the sum of all the individual masses mᵢ(t) at each of the times:

$$M = {\sum }_{i} {m}_{i} ( t )$$

The calculation is thus performed for a time t 30 minutes.

It will be noted that, in this approach, non-volatile oils are considered to have zero evaporation rates.

According to an advantageous embodiment, when other volatile lipophilic solvents (that do not correspond to the definition of the volatile solvents according to the invention) are present, the volatile solvent according to the invention should be present at least 30%, or better still 50%, by mass of the total sum of the lipophilic volatile solvents.

According to an advantageous embodiment, irrespective of whether or not the volatile solvent(s) according to the invention is (are) used as a mixture with other solvents (not corresponding to the definition of the volatile solvents according to the invention), the volatile solvent(s) according to the invention preferably represent(s) at least 2% by weight, or better still at least 5% by weight, relative to the total weight of the composition.

The term “volatile oil” or “volatile solvent” is intended to mean an oil (or nonaqueous medium) capable of evaporating on contact with the skin or with the keratin fibre, and more generally with the keratin material, in less than one hour, at ambient temperature and atmospheric pressure. The volatile oil is a volatile cosmetic oil, that is liquid at ambient temperature, having in particular a non-zero vapour pressure, at ambient temperature and atmospheric pressure, in particular having a vapour pressure ranging from 0.13 Pa to 40 000 Pa (10⁻³ to 300 mmHg), in particular ranging from 1.3 Pa to 13 000 Pa (0.01 to 100 mmHg), and more particularly ranging from 1.3 Pa to 8000 Pa (0.01 to 60 mmHg).

In the context of the present invention, the volatile oils which do not correspond to the definition of the volatile solvents according to the invention and which may be present in the composition are the oils for which the amount evaporated after 30 minutes under the conditions described above is greater than or equal to 0.07 mg/cm².

Among these volatile oils not in accordance with the definition of the volatile solvents according to the invention, mention may be made of cyclic or noncyclic silicone volatile oils, or non-silicone volatile oils, in particular chosen from hydrocarbon-based or fluorinated volatile oils, and mixtures thereof.

Among the “cyclic or noncyclic silicone volatile oils” mention may in particular be made of the linear oils having a viscosity 6 centistokes (6×10⁻⁶ m²/s), and having in particular from 3 to 10 silicon atoms, these silicones optionally comprising one or more alkyl or alkoxy groups containing 1 or 2 carbon atoms. In this category of silicone volatile oils that can be used in the invention, mention may in particular be made of octamethylcyclotetrasiloxane, decamethylcyclopenta-siloxane, dodecamethylcyclohexasiloxane, heptamethyl-hexyltrisiloxane, heptamethyloctyltrisiloxane, hexa-methyldisiloxane, octamethyltrisiloxane, decamethyl-tetrasiloxane and dodecamethylpentasiloxane, and mixtures thereof.

The noncyclic silicone volatile oils can also be chosen from linear or branched silicone volatile oils.

The hydrocarbon-based volatile oil not in accordance with the definition of the volatile solvents according to the invention can be chosen from hydrocarbon-based volatile oils containing from 8 to 16 carbon atoms, and mixtures thereof, and in particular C₈-C₁₆ branched alkanes, such as isoalkanes (also known as isoparaffins), isododecane (also known as 2,2,4,4,6-pentamethylheptane), isodecane or isohexa-decane, and, for example, the oils sold under the trade names Isopars® or Permethyls®.

Other hydrocarbon-based volatile oils, such as petroleum distillate, in particular those sold under the name “Shell Solt®” by the company Shell, may be used.

According to a variant of the invention, the composition of the present invention is free of cyclic or noncyclic silicone volatile oils, i.e. comprises less than 0.1% by weight of these cyclic or noncyclic silicone volatile oils, relative to the total weight of the composition.

According to another variant of the invention, the composition is free of cyclic silicone volatile oil, especially octamethylcyclotetrasiloxane, decamethyl-cyclopentasiloxane or dodecamethylcyclohexasiloxane, and in particular octamethylcyclotetrasiloxane, i.e. comprises less than 0.1% by weight of cyclic silicone oils relative to the total weight of the composition.

Preferably, when said volatile solvent according to the invention is used as a mixture with other volatile solvents not in accordance with the definition of the volatile solvents according to the invention, these other solvents are natural or of natural origin. In general, the various oils of the composition (volatile or non-volatile, and including the hydrocarbon-based volatile solvents according to the invention), the solid fatty substances or the other ingredients of the composition will preferably be natural or of natural origin.

The “natural” compounds are:
compounds of biological agricultural plant origin or wild-plant origin with carefully thought out sampling,
compounds of agricultural plant origin or originating from the kingdom Protista,
compounds of non-fossil mineral origin,
compounds of animal origin, preferably compounds secreted by animals (beeswax).

The compounds “of natural origin” are natural compounds having undergone conversions, it being possible for these conversions to be:

1) conversions which do not modify the composition of the starting material relative to its origin, other than possibly its water content. These conversions generate essentially modifications to the physical appearance of the ingredient relative to its origin. examples of conversions which fall into this category are:
crushing,
milling,
drying,
freeze-drying,
thermal preservation processes (sterilization in a hermetically sealed container, pasteurization),
pressurized preservation processes (Pascalization),
the addition of preserving agents of plant origin being accepted.
   2) processes aimed at extracting, in the case of ingredients of plant origin, a given fraction of the plant without breaking covalent chemical bonds, which encompasses the following processes:
expression,
pressing,
flash vacuum-expansion processes,
distillation,
water-extraction processes (decoction, infusion, maceration),
ethanol-extraction processes (including enfleurage),
extraction processes with supercritical CO₂,
the above extraction processes using microwave heating,
steam distillation,
purification processes,
purification processes based on the preceding technologies,
purification processes by passing over active charcoal, over oxides or over resin,
thermal preservation processes (sterilization in a hermetically sealed container, pasteurization),
winterization or cold clearing processes,
bioconversions applied to starting materials of plant origin and catalysed by non genetically modified organisms, and the original function of which corresponds to the targeted reaction,
pressurized preservation processes (Pascalization) or preservation processes using the addition of preserving agents of plant origin,
genetic extraction processes which do not fall into this category and also irradiation-based preservation processes.
   3) in the case of the non-fossil mineral materials, the conversion processes may be the following:
processes aimed at purifying or slightly modifying the starting material without significant modification of its crystalline structure or its
composition,
distillation,
purification processes (elimination of heavy metals, of organic compounds, etc.),
ion exchange processes,
purification processes by passing over active charcoal, over oxides or over resin,
thermal preservation processes,
pressurized preservation processes (Pascalization).
   4) conversions by chemical process generating a minor modification, and in particular with regard to compounds of plant origin:
extraction with an organic solvent (hexane, fluoro ethers, or the like),
hydrolysis,
esterification,
oxidation using oxygen as oxidant,
olefin hydrogenations,
hydrogenation of acids and of esters,
etherifications,
Guerbet reaction (intermolecular reaction between alcohols similar to a “cooking” process),
   and for the ingredients of non-fossil mineral origin: processes for obtaining materials by dissolution/reprecipitation of mineral species resulting in simple or structured oxides (zeolites, mesoporous compounds, etc.).
   5) conversions for a functionalization, in particular amination, nitration, silylation, carboxylation using catalysts of mineral or biological origin and also bioconversions by means of genetically modified organisms, the function of which may or may not correspond to the original reaction, and processes that give rise to the synthesis of oxide mixtures.

According to a first embodiment of the invention, a compound is in particular considered to be natural or of natural origin as defined above in points 4) or 5) when the amount by weight of a natural product or product of natural origin is greater than the amount by weight which does not correspond to this definition.

According to a second embodiment, a compound is in particular considered to be natural or of natural origin as defined above in points 4) or 5) when the number of carbon atoms of a natural compound or compound of natural origin is greater than the number of carbon atoms which do not correspond to this definition.

Thus, solvents that are not therefore considered to be natural compounds or compounds of natural origin include certain volatile solvents conventionally used in cosmetic compositions, such as isododecane, which is of mineral fossil origin (derived from petroleum chemistry) or cyclomethicone D5, which is a silicone compound prepared by chemical synthesis processes.

Advantageously, the compositions according to the invention are such that the volatile solvents which are not natural or of natural origin represent less than 20% by mass of the sum of the volatile solvents of the composition.

Preferably, the composition is such that the mixture of said volatile solvents and/or of said additional volatile oils and/or of fatty substances optionally present contains less than 2% by mass of non-natural compounds or compounds which are not of natural origin, relative to the mass of said mixture (it thus being possible for said mixture to be completely free of such compounds).

According to a specific embodiment, when said volatile solvent according to the invention is used in mixtures with other lipophilic volatile solvents which do not correspond to the definition of the volatile solvents according to the invention, the mixing must be carried out in such a way that the mixture of volatile solvents, or volatile fatty phase, in the composition according to the invention has an evaporation profile such that the mass of oil(s) evaporated after 30 minutes according to the conditions defined above is between 4.1 mg/cm² and 24 mg/cm².

Preferably, the volatile fatty phase comprising the volatile solvents according to the invention and, optionally, other volatile oils (that do not correspond to the definition of the volatile solvents according to the invention) represents a content ranging from 0.1% to 80% by weight, especially from 1% to 65% by weight, in particular from 10% to 50% by weight, relative to the total weight of the composition.

According to another aspect, a subject of the invention is also a cosmetic process for making up and/or caring for keratin materials, comprising at least the step of applying a composition according to the invention to the keratin materials.

Another subject of the invention is a process for preparing such make-up and/or care compositions.

### Physiologically Acceptable Medium

The term “physiologically acceptable medium” denotes a medium which is nontoxic and which can be applied to the skin, in particular of the body, the hands, the neck or the face, the lips and/or keratin fibres of human beings. The physiologically acceptable medium is generally suitable for the nature of the support on which the composition must be applied and also for the way in which the composition is intended to be packaged.

### Non-Volatile Oils

The composition according to the invention may also comprise at least one non-volatile oil. Said oil may in particular be chosen from non-volatile hydrocarbon-based and/or silicone and/or fluoro oils.

The term “non-volatile oil” is intended to mean an oil that remains on the skin or the keratin fibre, more generally on the keratin material, at ambient temperature and atmospheric pressure, for at least several hours and that in particular has a vapour pressure of less than 10⁻³ mmHg (0.13 Pa). A non-volatile oil can also be defined as having an evaporation rate such that, under the conditions defined above, the amount evaporated after 30 minutes is less than 0.07 mg/cm².

As non-volatile hydrocarbon-based oil, mention may in particular be made of:
hydrocarbon-based oils of plant origin, such as triglycerides consisting of fatty acid esters of glycerol, the fatty acids of which may have chain lengths ranging from C₄ to C₂₄, it being possible for the latter to be linear or branched, and saturated or unsaturated, such as triglycerides of heptanoic acid or octanoic acid; these oils are in particular wheatgerm oil, sunflower oil, grapeseed oil, sesame oil, maize oil, apricot oil, castor oil, Shea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin oil, sesame oil, marrow oil, rapeseed oil, blackcurrant oil, evening primrose oil, millet oil, barley oil, quinoa oil, rye oil, safflower oil, candlenut oil, passionflower oil or musk rose oil; or else caprylic/capric acid triglycerides, for instance those sold by the company Stéarineries Dubois or those sold under the names “Miglyol 810®”, “812®” and “818®” by the company Dynamit Nobel;
oils of animal origin, such as mink oil, turtle oil or perhydrosqualene;
synthetic ethers;
linear or branched hydrocarbons of mineral or synthetic origin, such as liquid paraffin or its derivatives, petroleum jelly, polydecenes, hydrogenated polyisobutenes such as Parleam® sold by the company Nippon Oil Fats, and squalene, and mixtures thereof;
esters of a fatty acid, in particular a fatty acid containing from 4 to 22 carbon atoms, and especially of octanoic acid, heptanoic acid, lanolic acid, oleic acid, lauric acid or stearic acid, such as propylene glycol dioctanoate, propylene glycol monoisostearate, polyglyceryl 2-diisostearate or neopentyl glycol diheptanoate;
synthetic esters, for instance oils of formula R₁COOR₂ in which R₁ represents a linear or branched fatty acid residue containing from 1 to 40 carbon atoms and R₂ represents a hydrocarbon-based chain, which is in particular branched, containing from 1 to 40 carbon atoms, on condition that R₁+R₂ is ≧11, for instance purcellin oil (cetostearyl octanoate), isononyl isononanoate, C₁₂ to C₁₅ alkyl benzoate, 2-ethylhexyl palmitate, 2-octyldodecyl stearate, 2-octyldodecyl erucate, isostearyl isostearate, 2-octyldodecyl benzoate, octanoates, decanoates or ricinoleates of alcohols or of polyalcohols, isopropyl myristate, isopropyl palmitate, butyl stearate, hexyl laurate, diisopropyl adipate, 2-ethylhexyl palmitate, 2-hexyldecyl laurate, 2-octyldecyl palmitate, 2-octyldodecyl myristate, 2-diethylhexyl succinate, diisostearyl malate or isododecyl neopentanoate;
hydroxylated esters, such as isostearyl lactate, octyl hydroxystearate, octyldodecyl hydroxy-stearate, diisostearyl malate, triisocetyl citrate, glyceryl triisostearate or diglyceryl triisostearate; diethylene glycol diisononanoate; and
pentaerythritol esters; esters of aromatic acids and of alcohols containing 4 to 22 carbon atoms, in particular tridecyl trimellitate;
fatty alcohols that are liquid at ambient temperature, with a branched and/or unsaturated carbon-based chain containing from 8 to 26 carbon atoms, for instance oleyl alcohol, linoleyl alcohol, linolenyl alcohol, isostearyl alcohol or octyldodecanol; C₈⁻C₂₆ higher fatty acids, such as oleic acid, linoleic acid, linolenic acid or isostearic acid;
and mixtures thereof.

The non-volatile silicone oils that can be used in the composition according to the invention may be non-volatile polydimethylsiloxanes (PDMSs), polydimethyl-siloxanes comprising alkyl or alkoxy groups, which are pendant and/or at the end of a silicone chain, these groups each containing from 2 to 24 carbon atoms, phenyl silicones, for instance phenyl trimethicones, phenyl dimethicones, phenyltrimethylsiloxydiphenyl-siloxanes, diphenyl dimethicones, diphenylmethyl-diphenyltrisiloxanes or 2-phenylethyl trimethylsiloxy-silicates.

According to one aspect of the invention, the composition is free of non-volatile oil, i.e. comprises less than 0.1% by weight of non-volatile oil relative to the total weight of the composition.

According to another aspect of the invention, the non-volatile oil may be present at a content ranging from 0.1% to 60% by weight, especially ranging from 0.5% to 50% by weight, and in particular ranging from 1% to 40% by weight, relative to the total weight of the composition.

### Solid Fatty Substances

The composition according to the invention may comprise, in particular when it is a lipstick or a foundation, at least one fatty substance that is solid at ambient temperature and at atmospheric pressure; it may be chosen from waxes, pasty fatty substances and gums, and mixtures thereof. This solid fatty substance may be present at a content ranging from 0.01% to 60%, especially from 0.1% to 50%, and in particular from 0.1% to 40% by weight, relative to the total weight of the composition.

Thus, the composition according to the invention may comprise at least one fatty compound that is pasty at ambient temperature.

For the purpose of the invention, the term “pasty fatty substance” is intended to mean fatty substances with a melting point ranging from 20 to 55° C., in particular 25 to 45° C., and/or a viscosity at 40° C. ranging from 0.1 to 40 Pa·s (1 to 400 poises), in particular 0.5 to 25 Pa·s, measured using a Contraves TV or Rheomat 80 viscometer, equipped with a spindle rotating at 60 Hz. Those skilled in the art can select the spindle for measuring the viscosity from the spindles MS-r3 and MS-r4, on the basis of their general knowledge, so as to be able to perform the measurement of the pasty compound tested.

More particularly, these fatty substances may be hydro-carbon-based compounds, optionally of polymeric type; they may also be chosen from silicone compounds; they may also be in the form of a mixture of hydrocarbon-based and/or silicone compounds. In the case of a mixture of various pasty fatty substances, hydrocarbon-based pasty compounds (mainly containing carbon and hydrogen atoms and possibly ester groups) are preferably used in major proportion.

Among the pasty compounds that may be used in the composition according to the invention, mention may be made of lanolins and lanolin derivatives, such as acetylated lanolins, oxypropylenated lanolins or isopropyl lanolate, with a viscosity of from 18 to 21 Pa·s, preferably 19 to 20.5 Pa·s, and/or a melting point of 30 to 55° C. and mixtures thereof. Use may also be made of esters of fatty acids or of fatty alcohols, in particular those containing 20 to 65 carbon atoms (melting point of the order of 20 to 35° C. and/or viscosity at 40° C. ranging from 0.1 to 40 Pa·s), for instance triisostearyl citrate or cetyl citrate; arachidyl propionate; polyvinyl laurate; cholesterol esters, for instance triglycerides of plant origin such as hydrogenated plant oils, viscous polyesters, and mixtures thereof. Triglycerides of plant origin that may be used include hydrogenated castor oil derivatives, such as “Thixinr®” from Rheox.

Mention may also be made of pasty silicone fatty substances such as high-molecular-weight polydimethyl-siloxanes (PDMSs), and in particular those with pendant chains of the alkyl or alkoxy type containing from 8 to carbon atoms, and a melting point of 20-55° C., for instance stearyl dimethicones, in particular those sold by the company Dow Corning under the trade names DC2503® and DC25514®, and mixtures thereof.

The pasty fatty substance may be present in the composition according to the invention at a content ranging from 0.01% to 50% by weight, especially ranging from 0.1% to 45% by weight, and in particular ranging from 0.2% to 30% by weight, relative to the total weight of the composition.

In the context of the present invention, the term “wax” is generally intended to mean a lipophilic compound that is solid at ambient temperature (25° C.), deformable or nondeformable, with a reversible solid/liquid change of state, having a melting point of greater than or equal to 30° C., which may be up to 200° C., and in particular up to 120° C.

By bringing the wax to the liquid state (melting), it is possible to render it miscible with oils and to form a microscopically homogeneous mixture, but when returning the temperature of the mixture to ambient temperature, recrystallization of the wax from the oils in the mixture is obtained.

In particular, the waxes suitable for the invention may have a melting point of greater than or equal to 45° C., and in particular greater than or equal to 55° C.

For the purpose of the invention, the melting point corresponds to the temperature of the most endothermic peak observed by thermal analysis (DSC) as described in ISO standard 11357-3; 1999. The melting point of the wax can be measured using a differential scanning calorimeter (DSC), for example the calorimeter sold under the name “MDSC 2920” by the company TA Instruments.

The measurement protocol is the following:

A sample of 5 mg of wax placed in a crucible is subjected to a first increase in temperature ranging from −20° C. to 100° C., at a heating rate of 10° C./minute, and is then cooled from 100° C. to −20° C. at a cooling rate of 10° C./minute, and, finally, subjected to a second increase in temperature ranging from −20° C. to 100° C. at a heating rate of 5° C./minute. During the second increase in temperature, the variation in the difference in power absorbed by the empty crucible and by the crucible containing the wax sample is measured as a function of temperature. The melting point of the compound is the value of the temperature corresponding to the top of the peak of the curve representing the variation in the difference in power absorbed as a function of temperature.

By way of waxes that may be used according to the invention, mention may be made of:
waxes of animal origin, such as beeswax, lanolin wax and lanolin derivatives, plant waxes such as carnauba wax, candelilla wax, ouricury wax, Japan wax, cocoa butter, cork fibre wax or sugarcane wax,
mineral waxes, for example paraffin wax, petroleum jelly wax, lignite wax, microcrystalline waxes or ozokerites,
synthetic waxes, among which are polyethylene waxes and waxes obtained by Fisher-Tropsch synthesis,
silicone waxes, in particular substituted linear polysiloxanes; mention may, for example, be made of silicone polyether waxes, alkyl dimethicones or alkoxy dimethicones containing from 16 to 45 carbon atoms, alkyl methicones such as the C₃₀-C₄₅ alkyl methicone sold under the trade name “AMS C 30” by Dow Corning,
hydrogenated oils that are solid at 25° C., such as hydrogenated castor oil, hydrogenated jojoba oil, hydrogenated palm oil, hydrogenated tallow or hydrogenated coconut oil, and fatty esters that are solid at 25° C., such as the C₂₀-C₄₀ alkyl stearate sold under the trade name “Kester Wax K82H” by the company Koster Keunen,
and/or mixtures thereof.

Preferably, polyethylene waxes, microcrystalline waxes, carnauba waxes, hydrogenated jojoba oil, candelilla waxes, beeswaxes and/or mixtures thereof will be used.

Aqueous and/or Water-Soluble Phase

The composition according to the invention may also comprise at least one aqueous phase containing water. The water may be a floral water such as cornflower water and/or a mineral water such as eau de Vittel, eau de Lucas or eau de La Roche Posay and/or a spring water.

The aqueous phase may also comprise organic solvents that are water-miscible (at 25° C.), for instance primary alcohols such as ethanol and isopropanol, glycols such as glycerol, propylene glycol, butylene glycol, dipropylene glycol, diethylene glycol, glycol ethers, C₁ to C₄ alkyl ethers of mono-, di- or tripropylene glycol, or mono-, di- or triethylene glycol, and mixtures thereof.

The composition may be an anhydrous composition, i.e. a composition containing less than 2% by weight of water, or even less than 0.5% of water, in particular free of water, the water not being added during the preparation of the composition, but corresponding to the residual water introduced by the ingredients mixed in.

### Particulate Phase

The composition of the invention may also comprise, in particular when it is a lipstick or a foundation, an additional particulate phase that may be present in a proportion of from 0.01% to 50% by weight, especially from 0.01% to 40% by weight, and in particular from 0.05% to 30% by weight, relative to the total weight of the composition.

The term “particulate phase” is intended to mean preferably ss and/or pearlescent agents and/or additional fillers, and/or mixtures thereof.

According to an embodiment, the composition of the invention comprise at least a pigment.

The term “pigments” should be understood to mean white or coloured, mineral or organic particles that are insoluble in the liquid hydrophilic phase and are intended to colour and/or opacify the composition. The term “fillers” should be understood to mean colourless or white, mineral or synthetic, lamellar or non-lamellar particles. The term “pearlescent agents” should be understood to mean iridescent particles, in particular produced by certain molluscs in their shell or alternatively which are synthesized.

The pigments may be present in the composition in a proportion of from 0.01% to 25% by weight, in particular from 0.01% to 20% by weight, and especially from 0.02% to 15% by weight, relative to the weight of the composition.

As mineral pigments that may be used in the invention, mention may be made of titanium oxide, zirconium oxide or cerium oxide and also zinc oxide, iron oxide or chromium oxide, ferric blue, manganese violet, ultramarine blue and chromium hydrate. Among the organic pigments that may be used in the invention, mention may be made of carbon black, the D & C pigments and lakes based on cochineal carmine, barium, strontium, calcium, or aluminium, or else the diketo pyrrolopyrroles (DPP) described in documents EP-A-542669, EP-A-787730, EP-A-787731 and WO-A-96/08537.

The pearlescent agents may be present in the composition in a proportion of from 0.01% to 25% by weight, especially from 0.01% to 15% by weight, and in particular from 0.02% to 10% by weight, relative to the total weight of the composition.

The pearlescent pigments may be chosen from white pearlescent pigments such as mica coated with titanium or with bismuth oxychloride, coloured pearlescent pigments such as titanium mica with iron oxides, titanium mica in particular with ferric blue or with chromium oxide, titanium mica with an organic pigment of the type mentioned above, and also pearlescent pigments based on bismuth oxychloride.

According to an embodiment, the composition of the invention comprises at least a filler.

The additional fillers may be present in a proportion of from 0.01% to 50% by weight, especially from 0.01% to 40% by weight, and in particular from 0.02% to 30% by weight, and even more particularly from 0.02% to 20% by weight, relative to the total weight of the composition.

They may in particular be spherical fillers such as, for example, talc, zinc stearate, mica, kaolin, polyamide (Nylon®) powders (Orgasol® from Atochem), polyethylene powders, tetrafluoroethylene polymer (Teflon®) powders, starch, boron nitride, polymeric microspheres such as those made of polyvinylidene chloride/acrylonitrile, for instance Expancel® (Nobel Industrie), or of acrylic acid copolymers (Polytrap® from the company Dow Corning), silicone resin microbeads (Tospearls® from Toshiba, for example), and elastomeric organopolysiloxanes.

The composition may also comprise water-soluble or liposoluble dyes at a content ranging from 0.01% to 6% by weight, relative to the total weight of the composition, in particular ranging from 0.01% to 3% by weight. The liposoluble dyes are, for example, Sudan Red, DC Red 17, DC Green 6, β-carotene, soybean oil, Sudan Brown, DC Yellow 11, DC Violet 2, DC Orange 5, and quinoline yellow. The water-soluble dyes are, for example, beetroot juice and methylene blue.

### Dyestuff

The composition according to the invention preferably comprises at least one dyestuff. The term “dyestuff” is intended to mean pigments and/or dyes and/or pearlescent agents, and/or mixtures thereof, as defined above.

The dyestuffs may be present in the composition at a content ranging from 0.01% to 50% by weight, relative to the total weight of the composition, preferably from 0.01% to 30% by weight.

### Additives

The composition according to the invention may also comprise any of the ingredients conventionally used in the fields concerned, and more especially in the cosmetics and dermatological field. These ingredients may in particular be chosen from polymers, in particular film-forming polymers, fixing polymers; surfactants; hair conditioners; opacifiers; fragrances; thickeners; gelling agents; hair dyes; silicone resins; silicone gums; preserving agents; antioxidants; active cosmetic agents; sunscreens; pH stabilizers; vitamins; moisturizers; antiperspirants; deodorants; self-tanning compounds, and mixtures thereof. The amounts of these various ingredients are those conventionally used in the fields concerned, and for example from 0.01% to 20% of the total weight of the composition.

Of course, those skilled in the art will take care to select this or these optional additional compound(s), and/or the amount thereof, in such a way that the advantageous properties of the composition according to the invention are not, or not substantially, impaired by the addition considered.

The composition of the invention may be obtained according to the preparation processes conventionally used in cosmetics or in dermatology.

The following examples are given by way of illustration and do not limit the invention.

### Formulation

The composition according to the invention may be in the form of a liquid, a paste, a solid, a foam or a spray. It may be an emulsion, in particular a direct or inverse emulsion, or else an anhydrous composition. It may also be in a two-phase form.

The composition finds a specific application as a body or facial care composition, a body or facial cleansing composition such as a shower gel, a bath gel or a make-up remover; a body or facial make-up composition such as a foundation, a lipstick, a lipcare product, a nailcare product, a mascara or an eyeliner; a fragrancing composition; a hair composition such as a hair dye composition or a composition for permanently reshaping the hair; an antisun composition; a deodorant composition; a hair cleansing or haircare composition such as a shampoo or a rinse-out or leave-in conditioner, a rinse-out composition to be applied before or after dyeing, bleaching, permanent-waving or hair straightening, or alternatively between the two steps of a permanent-waving or hair-straightening operation; a hair composition for holding the hairstyle, such as a styling lacquer, a gel, mousse or spray.

In particular, the composition according to the invention can be used for making up and/or caring for the skin, the lips and/or the keratin fibres of a human being.

According to a preferred aspect of the invention, the composition is in the form of lipsticks or complexion products, especially of the foundation type, or of a mascara.

When the composition according to the invention is of the mascara type, it may be applied uniformly or non-uniformly to the surface of the eyelashes, as a single coat or in the form of several superimposed coats. The composition according to the invention may then be more particularly intended for a mascara product comprising a reservoir, containing at least said mascara composition, and a system for applying said composition to the keratin fibres, for instance the eyelashes.

According to one aspect of the invention, this composition is in the form of a product cast as a stick or a dish, for instance lipsticks or lip balms, cast foundations, concealer products, complexion “correctors” and/or “enhancers” and eyeshadows or face powders.

For the purpose of the present invention, the term “cast composition” is intended to mean any cosmetic composition not having the capacity to flow under the action of its own weight, as opposed to “fluid” compositions.

These compositions may, where appropriate, have a pasty appearance at ambient temperature (25° C.). Thus, a cosmetic composition according to the invention may have a melting point or a thermal transition temperature such as a softening point of greater than 25° C., which may especially range from 25 to 85° C., or even from 30 to 60° C., and in particular from 30 to 45° C., and/or a hardness that may range from 0.001 to 0.5 MPa, and especially from 0.005 to 0.4 MPa.

The compositions according to this aspect of the invention, i.e. of cast type, have hardnesses, in particular when they are in stick form.

The aim of the examples which follow is to illustrate the subject of the present invention in a nonlimiting manner. The amounts are given as percentage by mass.

### EXAMPLE 1

Foundation in the form of a water-in-oil emulsion having the following composition:

| | | |
|---|---|---|
| Cetyl dimethicone copolyol (Abil EM 90 from | 3 | g |
| the company Goldschmidt) | | |
| Isostearyl diglyceryl succinate (Imwitor | 0.6 | g |
| 780K from the company Condea) | | |
| Pentane, 1,1′-oxybis- (dipentyl ether) | 23.58 | g |
| (42570-100 ML, Sigma Aldrich ®) | | |
| Mixture of pigments (hydrophobic iron oxides | 10 | g |
| and titanium oxides) | | |
| Bentone | 1.6 | g |
| Polyamide powder (Nylon-12 from Dupont de | 8 | g |
| Nemours) | | |
| Magnesium sulphate | 0.7 | g |
| Preserving agent | 0.45 | g |
| Fragrance | 0.5 | g |
| Water | qs 100 | g |

### EXAMPLE 2

Oil-in-water foundation having the following composition:

| | | |
|---|---|---|
| Hexyl acetate (148500010, AcrosOrganics ®) | 11 | g |
| Hydrogenated polyisobutene (Parleam, NOF | | |
| Corporation) | 5 | g |
| 2-Ethylhexyl palmitate | 11 | g |
| Glyceryl isostearate | 4 | g |
| Stearic acid | 2 | g |
| Triethanolamine | 1 | g |
| Polyamide powder (Nylon-12 from Dupont de | 5 | g |
| Nemours) | | |
| Mixture of pigments (iron oxides and titanium | 10 | g |
| oxides) | | |
| Carboxymethylcellulose | 0.2 | g |
| Propylene glycol | 5 | g |
| Glycerol | 2 | g |
| Fragrance | 0.5 | g |
| Preserving agents | 0.4 | g |
| Water | qs 100 | g |

### EXAMPLE 3

Lipstick having the following composition:

| | | |
|---|---|---|
| Polyethylene wax (Performalene 655, New Phase | 15 | g |
| Technologies) | | |
| Butyl butanoate (259590010, AcrosOrganics ®) | 70 | g |
| Dipropyl carbonate/(294934-25G, Aldrich ®) | 9 | g |
| DC Red No. 7 Calcium Lake (pigment) | 6 | g |

### EXAMPLE 4

Care cream having the following composition:

| | | |
|---|---|---|
| Fatty phase | | |
| Mixture of glyceryl monostearate and of | 2.5 | g |
| polyethylene glycol stearate 100 EO (50/50 by | | |
| weight) (Arlacel 165 from the company ICI) | | |
| Stearyl alcohol | 0.5 | g |
| Stearic acid | 1 | g |
| Hydrogenated polyisobutene (Parleam, NOF | 9 | g |
| Corporation) | | |
| 3-Nonanone (259519-250ML, Sigma Aldrich ®) | 4.2 | g |
| Aqueous phase | | |
| Crosslinked polyacrylic acid (Carbopol 980) | 1 | g |
| Triethanolamine | 0.03 | g |
| Preserving agent | 0.3 | g |
| Water | qs 100 | g |

### EXAMPLE 5

Make-up remover having the following composition:

| | | |
|---|---|---|
| Isopropyl palmitate | 8 | g |
| Decane, 2-ethoxy- | 8 | g |
| Stearyl alcohol | 8 | g |
| Sucrose stearate | 2 | g |
| Stearic acid | 0.3 | g |
| Sodium hydroxide | 0.06 | g |
| Glycerol | 5 | g |
| Carbopol | 0.2 | g |
| Water | qs 100 | g |

### EXAMPLE 6

Spray deodorant having the following composition:

| | | |
|---|---|---|
| Octanal (05608-500ML, Sigma-Aldrich ®) | 33 | g |
| PPG-14 butyl ether (Ucon Fluid AP - Amerchol) | 10 | g |
| Hydrogenated castor oil (Cutina HR - Cognis) | 4 | g |
| Talc | 2 | g |
| Aluminium hydrochloride (Micro Dry - Reheis) | 20 | g |
| Stearyl alcohol | 14 | g |
| PEG-8 distearate (PEG 400 distearate - | 2 | g |
| Stéarineries Dubois) | | |
| C12-15 alkyl benzoate (Finsolv TN - Witco) | 15 | g |

### EXAMPLE 7

Roll-on deodorant (emulsion) having the following composition:

| | | |
|---|---|---|
| Aluminium hydrochloride (50% solution) | 40 | g |
| (Chlorhydrol 50% USP) | | |
| Steareth 21 (Brij 721 - ICI) | 2 | g |
| Steareth 2 (Brij 2 - ICI) | 2 | g |
| PPG 15 stearyl ether (Arlamol E - ICI) | 1.5 | g |
| Dipropyl carbonate (294934-25G, Aldrich ®) | 3.5 | g |
| Water | qs 100 | g |

### EXAMPLE 8

Anhydrous antiperspirant aerosol having the following composition:

| | | |
|---|---|---|
| Stearalkonium bentonite sold under the name | 0.5 | g |
| Tixogel MP250 by Sud-Chemie Rheologicals, | | |
| United Catalysts Inc. | | |
| Aluminium hydrochloride | 7 | g |
| Isobutane | 80 | g |
| Triethyl citrate | 1.4 | g |
| Isopropyl palmitate | 3 | g |
| Pentyl propanoate (269470010, AcrosOrganics ®) | 8.1 | g |

### EXAMPLE 9

Suncream having the following composition:

| | | |
|---|---|---|
| Stearic acid | 0.95 | g |
| Glyceryl stearate (and) PEG-100 stearate | 2.00 | g |
| Cetyl alcohol (and) myristyl alcohol (and) | 0.50 | g |
| stearyl alcohol | | |
| Dimethicone | 0.50 | g |
| Phenoxyethanol (and) methylparaben (and) | 1.00 | g |
| ethylparaben (and) propylparaben (and) | | |
| isobutylparaben (and) butylparaben | | |
| C₁₂-C₁₅ alkyl benzoate | 8.00 | g |
| Ethylhexyl cocoate | 2.00 | g |
| Octocrylene | 7.00 | g |
| Ethylhexyl triazone | 1.00 | g |
| Butyl methoxydibenzoylmethane | 3.50 | g |
| Triethanolamine | 0.50 | g |
| Glycerol | 4.00 | g |
| Methylparaben (and) butylparaben (and) | 0.25 | g |
| ethylparaben (and) isobutylparaben (and) | | |
| propylparaben | | |
| Disodium EDTA | 0.10 | g |
| Water | 52.10 | g |
| Carbomer | 0.30 | g |
| Potassium cetyl phosphate | 1.00 | g |
| Triethanolamine | 0.30 | g |
| Titanium dioxide (and) aluminium hydroxide | 5.00 | g |
| (and) stearic acid | | |
| Hexyl acetate (148500010, AcrosOrganics ®) | 10.00 | g |

### EXAMPLE 10

Hairspray in a pump dispenser, having the following composition:

| | | |
|---|---|---|
| Octylacrylamide/acrylates/butylaminoethyl | 6 | g of AM |
| methacrylate amphoteric polymer (Amphomer ®, | | |
| National Starch) | | |
| Nonanal (76310-100 ML, Fluka ®) | 3 | g |
| Ethanol | qs 100 | g |

1-43. (canceled)

44. A cosmetic composition comprising, in a physiologically acceptable medium, at least one aprotic hydrocarbon-based volatile solvent comprising no more than one branch, wherein the volatility of the solvent is such that the amount evaporated in 30 minutes is from 4.1 mg/cm² to 24 mg/cm², and provided that
when the at least one aprotic hydrocarbon-based volatile solvent is an ether of formula R1-O—R2, the at least one aprotic hydrocarbon-based volatile solvent can comprise more than one branch,
wherein R1 and R2 are identical or different and represent a linear or branched hydrocarbon-based radical, with the proviso that:
when R1 and R2 are linear, they each comprise from 1 to 10 carbon atoms with 10≦R1+R2≦11; and
when R1 and/or R2 are branched, they each comprise from 1 to 12 carbon atoms with 10≦R1+R2≦13.

45. The composition according to claim 44, wherein the at least one aprotic hydrocarbon-based volatile solvent comprises at least one oxygen atom.

46. The composition according to claim 44, wherein the at least one aprotic hydrocarbon-based volatile solvent comprises from 7 to 13 carbon atoms.

47. The composition according to claim 44, wherein the at least one aprotic hydrocarbon-based volatile solvent is linear.

48. The composition according to claim 47, wherein the at least one aprotic hydrocarbon-based volatile solvent comprises from 7 to 11 carbon atoms.

49. The composition according to claim 44, wherein the at least one aprotic hydrocarbon-based volatile solvent is saturated.

50. The composition according to claim 44, wherein the at least one aprotic hydrocarbon-based volatile solvent is aliphatic.

51. The composition according to claim 44, wherein the at least one hydrocarbon-based volatile solvent is alkyl.

52. The composition according to claim 44, wherein the at least one aprotic hydrocarbon-based volatile solvent is lipophilic.

53. The composition according to claim 44, wherein the at least one aprotic hydrocarbon-based volatile solvent has a flashpoint ranging from 43 to 100° C.

54. The composition according to claim 53, wherein the at least one aprotic hydrocarbon-based volatile solvent has a flashpoint ranging from 45 and 80° C.

55. The composition according to claim 44, wherein the at least one aprotic hydrocarbon-based volatile solvent has a surface tension of less than 30 mN/m.

56. The composition according to claim 44, wherein the at least one aprotic hydrocarbon-based volatile solvent has a viscosity of less than 10 mPa·s.

57. The composition according to claim 44, wherein the at least one aprotic hydrocarbon-based volatile solvent is miscible with hydrogenated polyisobutene and isononyl isononanoate and/or has a water-miscibility of less than 5%.

58. The composition according to claim 44, wherein the at least one aprotic hydrocarbon-based volatile solvent is in liquid form at a temperature ranging from 4° C. to 100° C.

59. The composition according to claim 44, wherein the at least one aprotic hydrocarbon-based volatile solvent is chosen from esters, ethers, carbonic acid esters, ketones and aldehydes.

60. The composition according to claim 44, wherein the at least one aprotic hydrocarbon-based volatile solvent is an ester of formula R1COOR2, in which R1 represents a hydrogen atom or a linear or branched hydrocarbon-based radical, and R2 represents a linear or branched hydrocarbon-based radical, with the proviso that:
when R2 is a linear hydrocarbon-based radical and R1 is a hydrogen atom or a linear hydrocarbon-based radical, then 7≦R1+R2≦8; and
when at least one of R1 and R2 is a branched hydrocarbon-based radical, then 8≦R1+R2≦10.

61. The composition according to claim 44, wherein the at least one aprotic hydrocarbon-based volatile solvent is a ketone of formula R1—CO—R2, in which R1 and R2 are identical or different and represent a linear or branched hydrocarbon-based radical, with the proviso that:
when R1 and R2 are linear, they each comprise from 1 to 8 carbon atoms with 8≦R1+R2≦9; and
when R1 and/or R2 are branched, they each comprise from 1 to 10 carbon atoms with 9≦R1+R2≦11.

62. The composition according to claim 44, wherein the at least one aprotic hydrocarbon-based volatile solvent is an ether of formula R1-O—R2.

63. The composition according to claim 44, wherein the at least one aprotic hydrocarbon-based volatile solvent is a carbonic acid ester of formula R1-O—CO—O—R2, in which R1 and R2 are identical or different and represent a linear or branched hydrocarbon-based radical, with the proviso that:
when R1 and R2 are linear, they each comprise from 1 to 6 carbon atoms with 6≦R1+R2≦7; and
when R1 and/or R2 are branched, they each comprise from 1 to 8 carbon atoms with 6≦R1+R2≦9.

64. The composition according to claim 44, wherein the at least one aprotic hydrocarbon-based volatile solvent is an aldehyde of formula R1COH, in which R1 represents a linear or branched hydrocarbon-based radical, with the proviso that:
when R1 is linear, R1 comprises 7 or 8 carbon atoms; and
when R1 is branched, R1 comprises 8 to 10 carbon atoms.

65. The composition according to claim 44, further comprising at least one additional volatile oil different from the at least one aprotic hydrocarbon-based volatile solvent.

66. The composition according to claim 65, wherein the mixture of the at least one aprotic hydrocarbon-based volatile solvent and the at least one additional volatile oil has an evaporation rate such that the amount evaporated in 30 minutes ranges from 4.1 mg/cm² to 24 mg/cm².

67. The composition according to claim 65, wherein the at least one aprotic hydrocarbon-based volatile solvent and/or the at least one additional volatile oil are/is natural or of natural origin.

68. The composition according to claim 65, wherein the at least one aprotic hydrocarbon-based volatile solvent is nonnatural or is not of natural origin, and/or the at least one additional volatile oil is nonnatural or not of natural origin, and wherein the at least one aprotic hydrocarbon-based volatile solvent and/or the at least one additional volatile oil represent less than 20% by mass of the total sum of the volatile compounds of the composition.

69. The composition according to claim 65, wherein the mixture of the at least one aprotic hydrocarbon-based volatile solvents and/or of the at least one additional volatile oil and/or of at least one fatty substance optionally present comprises less than 2% by mass of nonnatural compounds or compounds that are not of natural origin, relative to the mass of the mixture.

70. The composition according to claim 65, wherein the at least one aprotic hydrocarbon-based volatile solvent represents at least 30% by mass of the total sum of the volatile compounds of the composition.

71. The composition according to claim 44, wherein the at least one aprotic hydrocarbon-based volatile solvent represents at least 2% by weight, relative to the total weight of the composition.

72. The composition according to claim 44, wherein the composition is in the form of a liquid, a paste, a solid, a foam or a spray.

73. The composition according to claim 44, wherein the composition is in the form of an emulsion or of an anhydrous composition.

74. The composition according to claim 44, wherein the composition is in the form of a body or facial care composition; a body or facial cleansing composition; a body or facial make-up composition; a fragrancing composition; a hair composition; an antisun composition; a deodorant composition; a hair cleansing or haircare composition; or a hair composition for holding the hairstyle.

75. The composition according to claim 44, wherein the composition is in the form of a product cast as a stick or a dish.

76. The composition according to claim 44, further comprising at least one non-volatile oil.

77. The composition according to claim 76, wherein the at least one non-volatile oil is chosen from natural oils and oils of natural origin.

78. The composition according to claim 44, further comprising at least one fatty substance that is solid at ambient temperature and atmospheric pressure.

79. The composition according to claim 78, wherein the at least one fatty substance is chosen from waxes, pasty fatty substances and gums.

80. The composition according to claim 44, further comprising an aqueous phase.

81. The composition according to claim 44, further comprising at least one particulate phase.

82. The composition according to claim 81, wherein the at least one particulate phase comprises pigments and/or fillers and/or pearlescent agents.

83. The composition according to claim 44, comprising at least one water-soluble or liposoluble dye.

84. The composition according to claim 44, further comprising at least one dyestuff.

85. The composition according to claim 44, further comprising at least one additive chosen from polymers; surfactants; hair conditioners; dyestuffs; pearlescent agents; opacifiers; organic solvents; fragrances; thickeners; gelling agents; waxes; pasty products; hair dyes; silicone resins; silicone gums; preserving agents; antioxidants; active cosmetic agents; sunscreens; pH stabilizers; vitamins; moisturizers; antiperspirants; deodorants; and self-tanning compounds.

86. The composition according to claim 85, wherein the at least one polymer is chosen from film-forming polymers and fixing polymers.

87. A cosmetic process for making up and/or caring for the skin, the lips and/or keratin fibres, comprising:
applying a cosmetic composition to the skin, the lips and/or the keratin fibres, wherein the cosmetic composition comprises, in a physiologically acceptable medium, at least one aprotic hydrocarbon-based volatile solvent comprising no more than one branch, wherein the volatility of the at least one aprotic hydrocarbon-based volatile solvent is such that the amount evaporated in 30 minutes is from 4.1 mg/cm² to 24 mg/cm², and provided that
when the at least one aprotic hydrocarbon-based volatile solvent is an ether of formula R1-O—R2, the at least one aprotic hydrocarbon-based volatile solvent comprise no more than one branch or more than one branch,
wherein R1 and R2 are identical or different and represent a linear or branched hydrocarbon-based radical, with the proviso that:
when R1 and R2 are linear, they each contain from 1 to 10 carbon atoms with 10≦R1+R2≦11; and
when R1 and/or R2 are branched, they each contain from 1 to 12 carbon atoms with 10≦R1+R2≦13.
